(19) United States
(12) Patent Application Publication
(10) Pub. No.: US 2022/0313426 A1
(43) Pub. Date: Oct. 6, 2022

(54) HYBRID EXPANDABLE DEVICE

(71) Applicant: The Foundry, LLC, Menlo Park, CA (US)

(72) Inventors: Hanson S. Gifford, III, Woodside, CA (US); Steven Kim, Los Altos, CA (US)

(21) Appl. No.: 17/596,544

(22) PCT Filed: Jun. 16, 2020
(86) PCT No.: PCT/US2020/037963

§ 371 Date: Dec. 13, 2021

Related U.S. Application Data
(60) Provisional application No. 62/862,433, filed on Jun. 17, 2019.

(51) Int. Cl. A61F 2/24

(52) U.S. Cl. CPC A61F 2/2418; A61F 2/2436; A61F 2210/0033; A61F 2250/0039; A61F 2250/0042

(57) ABSTRACT

The present technology is directed to the treatment of cardiac valves. Many embodiments of the present technology comprise an anchor member configured to be positioned at an implantation site proximate a native valve annulus. The anchor member may comprise an expandable structure having a first portion and a second portion. When the first portion is positioned at the implantation site at body temperature and released from a constrained delivery state, the first portion is configured to self-expand into apposition with tissue at or near the annulus to secure the anchor member at the implantation site. The second portion remains in a low-profile state at or around body temperature and is configured to expand into apposition with tissue at or near the annulus when heated above a second temperature greater than the first temperature and body temperature.

### CROSS-REFERENCE TO RELATED APPLICATION

The present application claims the benefit of priority to U.S. Provisional Application No. 62/862,433, filed Jun. 17, 2019, which is incorporated by reference herein in its entirety.

### TECHNICAL FIELD

The present technology relates to prosthetic heart valve devices. In particular, several embodiments are directed to prosthetic valves and devices for percutaneous repair and/or replacement of cardiac valves and associated systems and methods of use.

### BACKGROUND

Transcatheter aortic valve replacement (“TAVR”) is a relatively new, less invasive treatment for severe, symptomatic aortic stenosis. TAVR comprises delivering a prosthetic heart valve to the native annulus through a catheter, thereby avoiding open heart surgery and its associated risks. Expansion of the prosthetic heart valve from its low-profile state (for delivery through the catheter) to its expanded state at the native annulus typically occurs via self-expansion or balloon expansion of the prosthetic valve structure.

TAVR is advocated as an alternative to conventional surgical aortic valve replacement in patients who do not qualify for surgery. In the latter patient category, studies have demonstrated that TAVR significantly reduces all-cause mortality, repeat hospitalization, and cardiac symptoms compared with standard therapy, including balloon valvuloplasty. For patients at high risk for surgery, survival after TAVR was comparable to that of surgical replacement, albeit with different periprocedural risks. For example, recent studies show that treatment with self-expanding transcatheter valves is associated with greater risk of post-procedure left bundle-branch block (“LBBB”) and more frequent need for a new permanent pacemaker when compared with balloon-expandable valves. However, treatment with self-expanding valves is associated with better valve hemodynamics and a lower mean gradient. Accordingly, there is a need for improved transcatheter valve replacement and/or repair devices and delivery systems.

### SUMMARY

The present technology relates to prosthetic heart valve devices. In particular, several embodiments are directed to prosthetic valves and devices for percutaneous repair and/or replacement of cardiac valves and associated systems and methods of use. The subject technology is illustrated, for example, according to various aspects described below, including with reference to FIGS. 1-11. Various examples of aspects of the subject technology are described as numbered clauses (1, 2, 3, etc.) for convenience. These are provided as examples and do not limit the subject technology.

1. An anchor member configured to be positioned at a treatment site proximate a native valve annulus of a human patient, the anchor member comprising:
an expandable structure comprising a first portion and a second portion, each having a low-profile state and an expanded state, wherein, when the expandable structure is positioned at the treatment site at a first temperature of no greater than about 40° C. and released from a catheter:
   the first portion self-expands toward its expanded state and into apposition with tissue at or near the annulus to secure the anchor member at the treatment site, and
   the second portion remains in its low-profile state,
wherein the second portion of the expandable structure is configured to expand into apposition with tissue at or near the annulus when heated to a second temperature greater than the first temperature.

2. The anchor member of Clause 1, wherein the second portion is formed of a shape memory alloy (“SMA”) having an austenite finish temperature A_{f} that is: (a) greater than or equal to the second temperature, and (a) greater than body temperature.

3. The anchor member of Clause 1 or Clause 2, wherein the first temperature is from about 36° C. to about 40° C.

4. The anchor member of Clause 1 or Clause 2, wherein the first temperature is from about 36° C. to about 39° C.

5. The anchor member of Clause 1 or Clause 2, wherein the first temperature is from about 36° C. to about 38° C.

6. The anchor member of any one of Clauses 2 to 5, wherein the second temperature is no less than 37° C.

7. The anchor member of any one of Clauses 2 to 5, wherein the second temperature is no less than 38° C.

8. The anchor member of any one of Clauses 2 to 5, wherein the second temperature is no less than 39° C.

9. The anchor member of any one of Clauses 2 to 5, wherein the second temperature is no less than 40° C.

10. The anchor member of any one of Clauses 2 to 5, wherein the second temperature is from about 37° C. to about 40° C.

11. The anchor member of any one of Clauses 2 to 5, wherein the second temperature is from about 38° C. to about 40° C.

12. The anchor member of any one of Clauses 2 to 5, wherein the second temperature is from about 39° C. to about 40° C.

13. The anchor member of any one of the preceding Clauses, wherein the second portion is formed of an SMA having a martensite finish temperature M_{f} greater than or equal to the first temperature and an austenite finish temperature A_{f} less than or equal to the second temperature.

14. The anchor member of any one of the preceding Clauses, wherein the second portion is formed of an SMA having a martensite start temperature Mₛ greater than or equal to the first temperature and an austenite finish temperature A_{f} less than or equal to the second temperature.

15. The anchor member of any one of the preceding Clauses, wherein the second portion is formed of an SMA having:

a martensite finish temperature M_{f} greater than or equal to the first temperature,

a martensite start temperature Mₛ greater than or equal to the first temperature, and

an austenite finish temperature A_{f} less than or equal to the second temperature.

16. The anchor member of any one of Clauses 1 to 14 wherein the second portion is formed of an SMA having:

a martensite finish temperature M_{f} less than the first temperature,

a martensite start temperature Mₛ greater than or equal to the first temperature, and

an austenite finish temperature A_{f} less than or equal to the second temperature.

17. The anchor member of any one of the preceding Clauses, wherein the second portion has an austenite finish temperature A_{f} less than 37° C.

18. The anchor member of any one of the preceding Clauses, wherein the expandable structure is configured such that, when implanted at the native valve annulus, the second portion is upstream of the first portion.

19. The anchor member of any one of the preceding Clauses, wherein the expandable structure is configured such that, when implanted at or near a native aortic valve annulus, (a) at least a portion of the first portion is positioned within the aorta and, (b) at least a portion of the second portion is positioned within the left ventricle.

20. The anchor member of any one of the preceding Clauses, wherein the expandable structure is configured such that, when implanted at or near a native aortic valve annulus, no portion of the first portion is pressing outwardly against the left ventricle.

21. The anchor member of any one of the preceding Clauses, wherein the expandable structure is configured such that, when implanted at or near an annulus of an aortic valve of the patient, no portion of the first portion is distal of the annulus.

22. The anchor member of any one of the preceding Clauses, wherein, when the expandable structure is implanted at or near a native aortic valve annulus such that both the first and second portions are expanded and in contact with adjacent tissue, the first portion presses outwardly against adjacent tissue with greater force than the second portion presses outwardly against adjacent tissue.

23. The anchor member of any one of the preceding Clauses, wherein the native valve is an aortic valve.

24. The anchor member of any one of the preceding Clauses, wherein the native valve is a mitral valve.

25. The anchor member of any one of the preceding Clauses, wherein the second portion is heat-expandable.

26. The anchor member of any one of the preceding Clauses, wherein the second portion does not self-expand at or below the second temperature.

27. The anchor member of any one of the preceding Clauses, wherein the second portion is an SMA.

28. The anchor member of any one of the preceding Clauses, wherein the second portion is nitinol.

29. The anchor member of any one of the preceding Clauses, wherein each of the first portion and the second portion comprises an SMA.

30. The anchor member of any one of the preceding Clauses, wherein the first portion includes a first SMA and the second portion includes a second SMA different than the first SMA.

31. The anchor member of any one of the preceding Clauses, wherein the first portion includes a first SMA comprising a first metal and a second metal and the second portion includes a second SMA comprising the first and second metals, wherein a proportion of the first metal to the second metal in the first SMA is different than a proportion of the first metal to the second metal in the second SMA.

32. The anchor member of any one of the preceding Clauses, wherein the expandable structure is formed of a plurality of interconnected struts.

33. The anchor member of any one of the preceding Clauses, wherein, at least when the anchoring member is in an unconstrained, expanded state, an angle between adjacent struts in the first portion is less than an angle between adjacent struts in the second portion.

34. The anchor member of any one of the preceding Clauses, wherein the struts comprising the second portion have a generally square-shaped cross-section.

35. The anchor member of any one of the preceding Clauses, wherein the struts comprising the second portion have a thickness to width ratio of about 1.

36. The anchor member of any one of the preceding Clauses, wherein the struts comprising the first portion have a different cross-sectional shape than the struts comprising the second portion.

37. The anchor member of any one of the preceding Clauses, wherein, when in the expanded state, the second portion is more rigid than the first portion.

38. The anchor member of any one of the preceding Clauses, wherein the first portion is configured to exert a continuous spring force against adjacent tissue while the device is implanted.

39. An expandable device configured to be positioned at an implantation site proximate a native valve annulus, the expandable device comprising:
an anchoring member comprising:
   an expandable structure comprising a first portion and a second portion,
   wherein, when the first portion is positioned at the implantation site at a first temperature and released from a constrained delivery state, the first portion is configured to self-expand into apposition with tissue at or near the annulus to secure the anchor member at the implantation site, and
   wherein the second portion remains in a low-profile state at the first temperature and is configured to expand into apposition with tissue at or near the annulus when heated to a second temperature greater than the first temperature secure the anchor member at the implantation site; and
a prosthetic valve configured to be carried by, mounted within, or coupled to the anchoring member.

40. The expandable device of any one of the preceding Clauses, wherein the anchoring member comprises any one of the devices of Clauses 1 to 38.

41. A system for treating a native cardiac valve of a human patient, the system comprising:
a sheath;
an anchoring member configured to be delivered through the sheath to a treatment site proximate a native valve annulus, the anchoring member comprising:
   an expandable structure comprising a first portion and a second portion,
   wherein, when the first portion is positioned at the treatment site at a first temperature and released from a constrained delivery state, the first portion is configured to self-expand into apposition with tissue at or near the annulus to secure the anchor member at the treatment site, and
   wherein the second portion remains in a low-profile state at the first temperature and is configured to expand into apposition with tissue at or near the annulus when heated to a second temperature greater than the first temperature;
a prosthetic valve configured to be carried by, mounted within, or coupled to the anchoring member;
an elongated member having a proximal end portion configured to be positioned at an extracorporeal location during implantation of the expandable structure and a distal end portion configured to be delivered through the sheath to the treatment site; and a heating element carried by the distal end portion of the elongated member, wherein the heating element is configured to facilitate heating of the second portion to the second temperature.

42. The system of any one of the preceding Clauses, wherein the heating element is a plurality of openings in the distal end portion of the elongated member, and wherein the system further comprises a fluid source coupled to the proximal end portion of the elongated member and configured to deliver heated fluid through a lumen extending through the elongated member and through the openings to a vicinity of the second portion.

43. The system of any one of the preceding Clauses, wherein the heating element is a balloon carried by the distal end portion of the elongated member, and wherein the system further comprises a fluid source coupled to the proximal end portion of the elongated member and configured to deliver heated fluid to the balloon through a lumen extending through the elongated member.

44. The system of any one of the preceding Clauses, wherein the heating element is an expandable basket carried by the distal end portion of the elongated member, and wherein the system further comprises a power source coupled to the elongated member, and wherein at least a portion of the elongated member is electrically conductive such that, when the power source is activated, the elongated member transfers energy to the basket, thereby heating and expanding the basket.

45. The system of any one of the preceding Clauses, wherein the heating element is an electrode that is in direct contact with the second portion, and wherein applying current to the elongated member passes current through the second portion, thereby heating the second portion and causing it to expand.

46. The system of any one of the preceding Clauses, wherein the anchoring member is any one of the anchoring members of Clauses 1 to 38.

47. A method of treating a native cardiac valve of a human patient, the method comprising:
delivering an anchoring member to a native valve region, the anchoring member comprising an expandable structure formed of a plurality of struts, wherein the expandable structure comprises a self-expandable first portion and a heat-expandable second portion;
releasing the first portion from a delivery sheath, thereby allowing the first portion to self-expand into apposition with tissue at the native valve region; and
heating the second portion to a temperature greater than 37° C. to transform the second portion from a low-profile delivery state to an expanded state in which the second portion is in contact with tissue at the native valve region.

48. The method of any one of the preceding Clauses, wherein releasing the first portion includes releasing the first portion such that the first portion self-expands into apposition with tissue downstream of the annulus.

49. The method of any one of the preceding Clauses, wherein the valve is the aortic valve and the method further comprises positioning at least a portion of the first portion in contact with the aortic wall, and positioning at least a portion of the second portion in contact with the left ventricular wall.

50. The method of any one of the preceding Clauses, wherein the cardiac valve is an aortic valve.

51. The method of any one of the preceding Clauses, wherein the cardiac valve is a mitral valve.

52. The method of any one of the preceding Clauses, wherein releasing the first portion occurs before heating the second portion.

53. The method of any one of the preceding Clauses, wherein releasing the first portion occurs at least partially while heating the second portion.

54. The method of any one of the preceding Clauses, wherein releasing the first portion occurs after heating the second portion.

55. The method of any one of the preceding Clauses, wherein heating the second portion includes infusing a heated fluid to the distal portion while positioned at the native valve annulus.

56. The method of any one of the preceding Clauses, wherein heating the second portion includes positioning a balloon at least partially within a lumen of the second portion and inflating the balloon with a heated fluid.

57. The method of any one of the preceding Clauses, wherein heating the second portion includes positioning an expandable member at least partially within a lumen of the second portion and heating the expandable member.

58. The method of any one of the preceding Clauses, wherein heating the second portion includes delivering current through the second portion.

59. The method of any one of the preceding Clauses, wherein the second portion does not self-expand at or below the first temperature.

60. The method of any one of the preceding Clauses, wherein the second portion is an SMA.

61. The method of any one of the preceding Clauses, wherein the second portion is nitinol.

62. The method of any one of the preceding Clauses, wherein each of the first portion and the second portion comprises an SMA.

63. The method of any one of the preceding Clauses, wherein the first portion includes a first SMA and the second portion includes a second SMA different than the first SMA.

64. The method of any one of the preceding Clauses, wherein the first portion includes a first SMA comprising a first metal and a second metal and the second portion includes a second SMA comprising the first and second metals, wherein a proportion of the first metal to the second metal in the first SMA is different than a proportion of the first metal to the second metal in the second SMA.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1 shows an expandable device of the present technology implanted at a native aortic valve.

FIG. 2A is a hysteresis curve illustrating how shape memory alloys with different characteristic temperatures behave at 37° C.

FIGS. 2B and 2C are hysteresis curves illustrating how the expandable device of the present technology behaves at certain temperatures.

FIGS. 3A-3C illustrate a method of implanting an expandable device of the present technology using a retrograde approach.

FIGS. 4A-7C illustrate various devices, systems, and methods for delivering thermal energy to an expandable device positioned at a native valve annulus in accordance with the present technology.

FIGS. 8 and 9 show strut angles of conventional self-expanding and balloon-expandable stents, respectively.

FIGS. 10A and 10B are cross-sections of struts of typical self-expanding and balloon-expandable devices, respectively.

FIG. 11 is a table showing conventional self-expanding and balloon-expandable devices.

### DETAILED DESCRIPTION

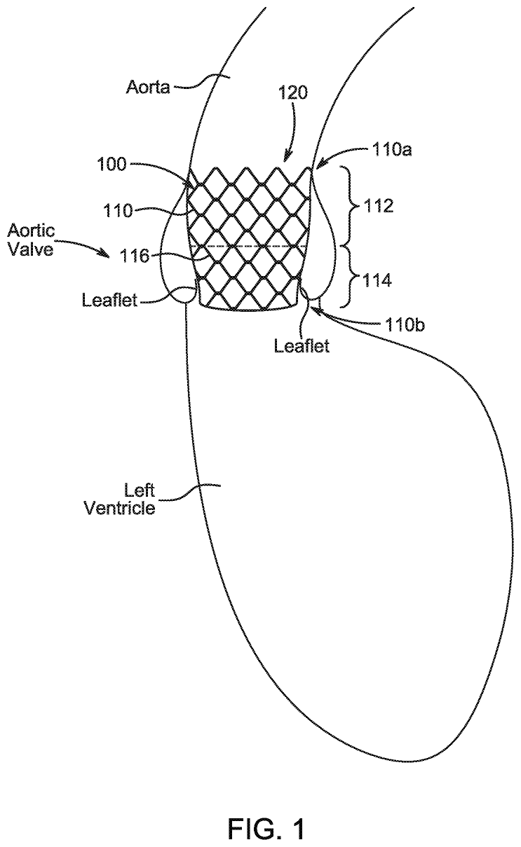

Specific details of several embodiments of the technology are described below with reference to FIGS. 1-11. Although many of the embodiments are described below with respect to devices, systems, and methods for percutaneous replacement of a native aortic valve, other applications and other embodiments in addition to those described herein are within the scope of the technology, such as devices, systems, and methods for percutaneous replacement of a native mitral valve. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described below with reference to FIGS. 1-11.

With regard to the terms “distal” and “proximal” within this description, unless otherwise specified, the terms can reference a relative position of the portions of a prosthetic valve device and/or an associated delivery device with reference to an operator and/or a location in the vasculature or heart. For example, in referring to a delivery catheter suitable to deliver and position various prosthetic valve devices described herein, “proximal” can refer to a position closer to the operator of the device or an incision into the vasculature, and “distal” can refer to a position that is more distant from the operator of the device or further from the incision along the vasculature (e.g., the end of the catheter).

As previously mentioned, conventional TAVR devices are typically self-expanding or balloon-expandable, each accompanied by unique advantages and disadvantages. Self-expanding structures, for example, do not require a balloon or other element to facilitate expansion, and thus can be delivered (a) within smaller catheters than balloon-expandable structures, and (b) without occluding blood flow at the treatment site during the procedure. Procedures employing a balloon expandable structure require temporarily reducing the patient's cardiac output while the balloon is expanded. This reduction is frequently achieved by rapid ventricular pacing (“RVP”), and some evidence suggests that patients undergoing RVP during a TAVR procedure may have a greater likelihood of in-hospital mortality and long-term mortality as compared to patients undergoing TAVR without RVP. Self-expandable structures, however, also come with certain risks. For example, recent studies show that treatment with self-expanding valve structures is associated with greater risk of post-procedure left bundle-branch block (“LBBB”) and more frequent need for a new permanent pacemaker (as compared with balloon-expandable valves). Increased incidence of LBBB is believed to be caused at least in part by the constant spring force exerted by self-expanding stents against the portion of the left ventricular wall corresponding to the location of the left bundle branch (“LBB”) of the heart's electrical conduction system. Constant outward pressure against the LBB can block electrical signals from the LBB to the rest of the heart (causing LBBB), thereby disrupting the normal contraction patterns of the heart and requiring implantation of a permanent pacemaker.

To address these challenges, the expandable devices of the present technology comprise an expandable hybrid stent structure that leverages the multi-phase properties of shape memory alloys (such as nitinol) to impart the clinical benefits provided by self-expanding and balloon-expandable structures while reducing or eliminating their attendant downsides. FIG. 1, for example, depicts an expandable device 100 (or “device 100”) of the present technology implanted in the native aortic valve region of the heart. As shown in FIG. 1, the device 100 can include an anchoring member 110 and a prosthetic valve 120 (not visible) coupled to, mounted within, or otherwise carried by the anchoring member 110. In some embodiments, the device 100 comprises only the anchoring member 110 and does not include the prosthetic valve 120. The anchoring member 110 can be movable between a low-profile delivery configuration (not shown) and a deployed configuration (FIG. 1). In the delivery configuration, the expandable device 100 has a low profile suitable for delivery through a small-diameter guide catheter configured to be positioned in the heart via trans-septal, retrograde, or trans-apical approaches.

As shown in FIG. 1, the anchoring member 110 may comprise a stent-like structure formed of a plurality of interconnected struts 116 (only one labeled for ease of illustration) surrounding a central lumen. The anchoring member 110 may have a first end portion 110a, a second end portion 110b, and a length extending therebetween along the longitudinal axis of the anchoring member 110. The anchoring member 110 is configured to be deployed at a native valve annulus (such as the aortic or mitral valve annulus) such that the second end portion 110b is positioned upstream or distal of the first end portion 110a.

The anchoring member 110 may include a self-expandable first portion 112 and a heat-expandable (or balloon-expandable) second portion 114. When implanted at the native valve region of an aortic valve, the anchoring member 110 is configured to be positioned such that the first, self-expanding portion 112 is positioned downstream of the second, thermally-expandable portion 114. As such, the first, self-expanding portion 112 may be positioned in apposition with an inner surface of the aortic wall, annular tissue, and/or the native valve leaflets. Preferably, the self-expanding portion 112 does not press outwardly against any portion of the left ventricular wall or other tissue that coincides with the location of the LBB or other conduction units of the heart. The second, heat-expandable portion 114 of the anchoring member 110 may be positioned in apposition with the aortic wall, the native leaflets, annular tissue, and/or the left ventricular wall. In some embodiments, the entirety of the heat-expandable portion 114 is positioned upstream of the annulus and within the left ventricle. As discussed elsewhere herein, the heat-expandable portion 114 does not exert a continuous outward force against the left ventricular wall, thereby eliminating or reducing disruption of the heart's electrical conduction system (as compared to a similarly positioned self-expanding structure).

In several aspects of the technology, all or a portion of the self-expanding portion 112 and all or a portion of the heat-expandable portion 114 of the anchoring member 110 may be formed of a shape memory alloy (“SMA”). SMAs are a unique class of metal alloys that can recover their “remembered” shapes when heated above a certain temperature. SMAs have two stable phases: (a) austenite, or the high-temperature phase in which the SMA is more rigid and superelastic, and (b) martensite, or the low-temperature phase in which the SMA is easily deformable. SMAs have four characteristic temperatures: (a) a martensitic start temperature (Mₛ), or the temperature at which the material undergoing cooling starts transforming from austenite to martensite, (b) a martensitic finish temperature (M_{f}), at which the transformation of the SMA is complete and the material is fully in the martensite phase, (c) an austenite start temperature (Aₛ) at which an SMA undergoing heating initiates the reverse transformation (austenite to martensite); and (d) an austenite finish temperature (A_{f}) at which the reverse phase transformation is completed and the SMA is in the austenite phase.

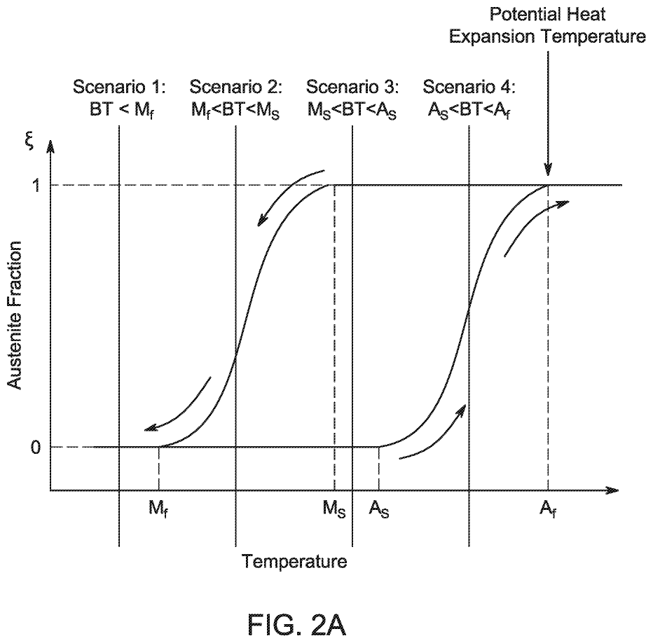

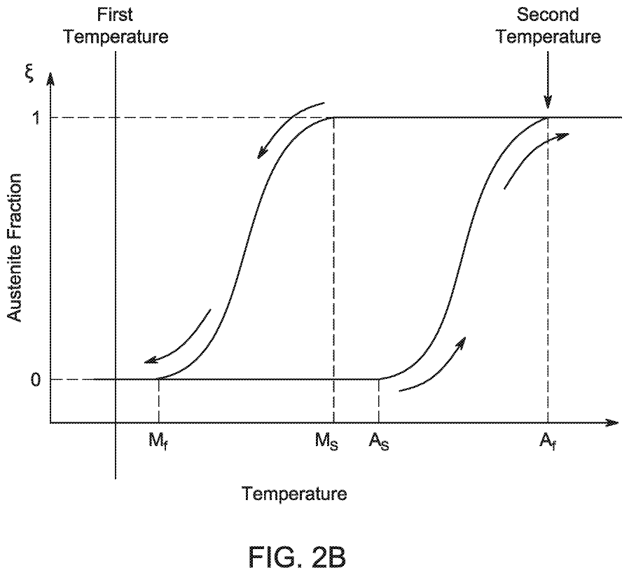

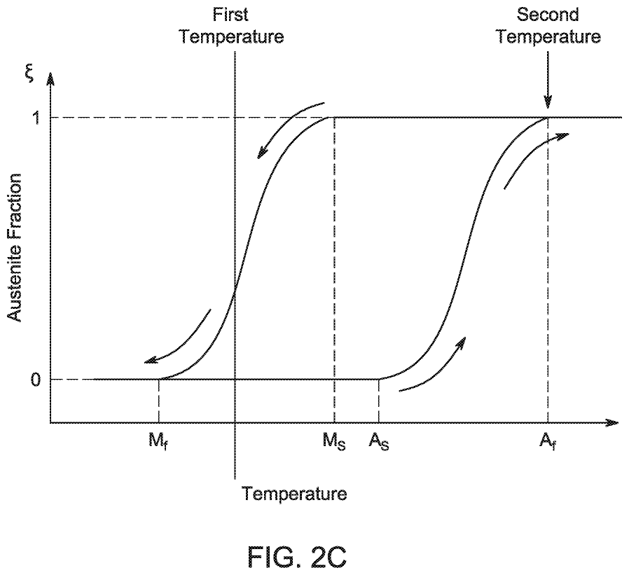

FIG. 2A is a hysteresis curve illustrating how SMAs with different characteristic temperatures behave at about 37° C. (e.g., average body temperature of a human patient). It will be appreciated that the discussion below equally applies to reference temperatures other than about 37° C. depending on the SMA. Referring to FIG. 2A, the first scenario depicts an SMA having an M_{f} greater than about 37° C. In this scenario, full expansion at A_{f} occurs at higher temperatures (relative to scenarios 2-4); however, when the SMA cools to 37° C. it will be fully martensitic. The second scenario depicts an SMA having an Mₛ greater than 37° C. but an M_{f} less than 37° C. In this scenario, the A_{f} is lower than the A_{f} in the first scenario, and thus full expansion occurs at lower temperatures than in the first scenario. When cooled to 37° C., this SMA will be in a hybrid martensite/austenite phase. The third scenario depicts an SMA having an Aₛ greater than 37° C. but an Mₛ less than 37° C. Here, the A_{f} is lower than the A_{f} in the first and second scenarios, and thus full expansion occurs at lower temperatures than in the first and second scenarios. When the SMA of the third scenario cools to 37° C., the SMA will still be full austenite. The fourth scenario depicts an SMA having an A_{f} greater than 37° C. and an Aₛ less than 37° C. Thus, full expansion of this SMA occurs at temperatures very close (but still greater than) 37° C., and the SMA will remain in its fully-expanded, austenite phase at 37° C.

In some embodiments, all or a portion of the self-expanding portion 112 of the anchoring member 110 may be formed of an SMA or other material configured to self-expand to a preset, expanded shape at a temperature below 36° C. For example, the self-expanding portion 112 may have an austenite finish temperature A_{f} less than 36° C. The self-expanding portion 112 is generally slightly oversized such that, while implanted, the self-expanding portion 112 exerts a continuous spring force against adjacent tissue to secure the expandable device 100 at the treatment site.

According to several embodiments of the present technology, all or a portion of the heat-expandable portion 114 may be formed, at least in part, of an SMA meeting the criteria of the first or second scenarios depicted in FIG. 2A. Such an SMA includes, for example, nitinol. It will be appreciated that other SMA's meeting the criteria detailed herein may be used with any of the heat-expandable portions 114, expandable devices, and/or anchoring members of the present technology.

As depicted in FIG. 2B, in some embodiments, all or a portion of the heat-expandable portion 114 may have an M_{f} greater than or equal to a first temperature and an A_{f} less or equal to a second temperature greater than the first temperature. The second temperature, for example, may be greater than body temperature. Unless expressly stated otherwise, “body temperature” as used herein refers to a temperature of about 36° C. to about 40° C., about 36° C. to about 39° C., or about 36° C. to about 38° C., or no more than 40° C. The A_{f} of the heat-expandable portion 114 and/or the second temperature may be 37° C. or greater, 38° C. or greater, 39° C. or greater, 40° C. or greater, from about 37° C. to about 40° C., from about 38° C. to about 40° C., or from about 39° C. to about 40° C.

As such, when the heat-expandable portion 114 is positioned in a delivery sheath and/or body at or below the first temperature, the heat-expandable portion 114 is in a low-profile, martensitic state. Thus, during delivery through the delivery sheath, the heat-expandable portion 114 exerts little or no outward force against an inner lumen wall of the delivery sheath, thereby improving ease of delivery through the sheath (relative to a self-expanding structure). When the heat-expandable portion 114 is released from the delivery sheath and heated to or above the second temperature, the heat-expandable portion 114 fully expands to its “remembered” or more rigid austenite state. When the heat-expandable portion 114 is subsequently cooled from the second temperature/austenite state to a temperature at or below the first temperature, the heat-expandable portion 114 transforms to its more malleable, fully martensite state. The heat-expandable portion 114 remains in contact with adjacent tissue at the treatment site in this fully martensite state. However, unlike a typical self-expanding structure, the heat-expandable portion 114 does not exert a constant outward pressure against the adjacent tissue and thus reduces or eliminates the potential for LBBB development as compared to similarly positioned, conventional, self-expanding stents.

As depicted in FIG. 2C, in some embodiments, all or a portion of the heat-expandable portion 114 may have an M_{f} less than a first temperature, an Mₛ greater than or equal to the first temperature, and an A_{f} less or equal to a second temperature greater than the first temperature. As such, when the heat-expandable portion 114 is positioned in a delivery sheath and/or body at or below the first temperature, the heat-expandable portion 114 is in a fully martensitic state. Thus, during delivery through the delivery sheath, the heat-expandable portion 114 exerts little to no outward force against an inner lumen wall of the delivery sheath, thereby improving ease of delivery through the sheath (relative to a self-expanding structure). When the heat-expandable portion 114 is heated to or above the second temperature, the heat-expandable portion 114 fully expands to its “remembered” or more rigid austenite state. When the heat-expandable portion 114 is subsequently cooled from the second temperature/austenite state to a temperature at or below the first temperature, the heat-expandable portion 114 transforms to its more malleable, partially-martensite state. The heat-expandable portion 114 remains in contact with adjacent tissue at the treatment site in this partially-martensite state but, unlike a typical self-expanding structure, the heat-expandable portion 114 exerts only a slight pressure against the adjacent tissue. This embodiment may be preferred over the fully-martensitic embodiment detailed with the respect to FIG. 2B in cases where a stiffer/more rigid implant is desired.

In any of the embodiments herein, the first temperature may approximate an upper limit of human body temperature, such as 40° C., and the second temperature may be greater than 40° C. In some embodiments, the first temperature is from about 36° C. to about 40° C., from about 36° C. to about 39° C., or from about 36° C. to about 38° C., and the second temperature is no less than 37° C., no less than 38° C., no less than 39° C., or no less than 40° C., from about 37° C. to about 40° C., from about 38° C. to about 40° C., or from about 39° C. to about 40° C.

The self-expanding and heat-expandable portions 112 and 114 may comprise the entirety of the anchoring member 110, or only a portion of the anchoring member 110. In some embodiments, the self-expanding portion 112 and the heat-expandable portion 114 abut one another along the length of the anchoring member 110, and in some embodiments the self-expanding portion 112 and the heat-expandable portion 114 are spaced apart along the length of the anchoring member 110, or mechanically coupled to one another at a joint. The self-expanding and heat-expandable portions 112 and 114 may have the same or different lengths and/or may extend around all or a portion of the circumference of the anchoring member 110. All or a portion of the self-expanding portion 112 may be radially aligned or overlap with all or a portion of the heat-expandable portion 114.

All or a portion of the self-expanding portion 112 may be formed of a first alloy and all or a portion of the heat-expandable portion 114 may be formed of a second alloy different than the first alloy. The first alloy may be an SMA. In some embodiments, the first alloy is not an SMA. In some embodiments, the first and second alloys are both SMAs. In such embodiments, each of the first and second alloys may be comprised of a mixture of the same types of metals but in different proportions such that the first and second alloys have different transition temperatures. For example, in some embodiments the first alloy and second alloys may comprise nitinol, but the first alloy may have a proportion of nickel and titanium that is different than the proportion of nickel and titanium in the second alloy. In some embodiments, the first and second alloys are both SMAs but have at least one metal that is different. In some embodiments, the first and second alloys are both SMAs but do not have any metals in common.

In those embodiments where two separate and distinct stents (e.g., one for the heat-expandable portion 114 and one for the self-expanding portion 112) are joined to create the expandable device and/or anchoring member having different transition temperatures, a variety of methods may be used to couple the two stents so as to ensure structural integrity and still minimize overall compressed diameter. For example, the stents may be connected with rivets, sutures, or other connectors. It may be preferable to make these connections proximal to valve, to avoid any increase in the overall diameter of the stent-valve during delivery. The separate stents may be positioned end-to-end such their adjacent ends abut and are in contact with one another but do not axially overlap. In some embodiments, the separate stents may overlap one another along a portion of their lengths. In some embodiments, the adjacent ends of the separate stents may be spaced apart along the length of the stent. In those embodiments, a coupling element and/or an additional structural component may span the distance between the two stents.

In some embodiments, heat-expandable portion 114 and the self-expanding portion 112 may be manufactured from a single piece of SMA to form a unitary structure. In such embodiments, the heat-expandable portion 114 and the self-expanding 112 portion are integral with one another. The transition temperatures of the two sections might be differentiated via careful heat treatment, where one section is kept cooler while the other section is annealed for an extra period of time. Achieving this may involve fixturing the stent with heat-sinks or cooled elements to keep one section cool while the other is heated. Precise fixturing might make the transition from one stent section to the other more specific.

Any of the anchoring members and/or expandable devices (or portions thereof) disclosed herein may be formed of a laser-cut tube, a braid formed of a plurality of filaments, a weave, and other suitable mesh structures. As used herein, “stent” refers to any of the foregoing mesh structures.

In some embodiments, the expandable device and/or anchoring member may comprise more than two discrete structures (e.g., three stents, four stents, etc.).

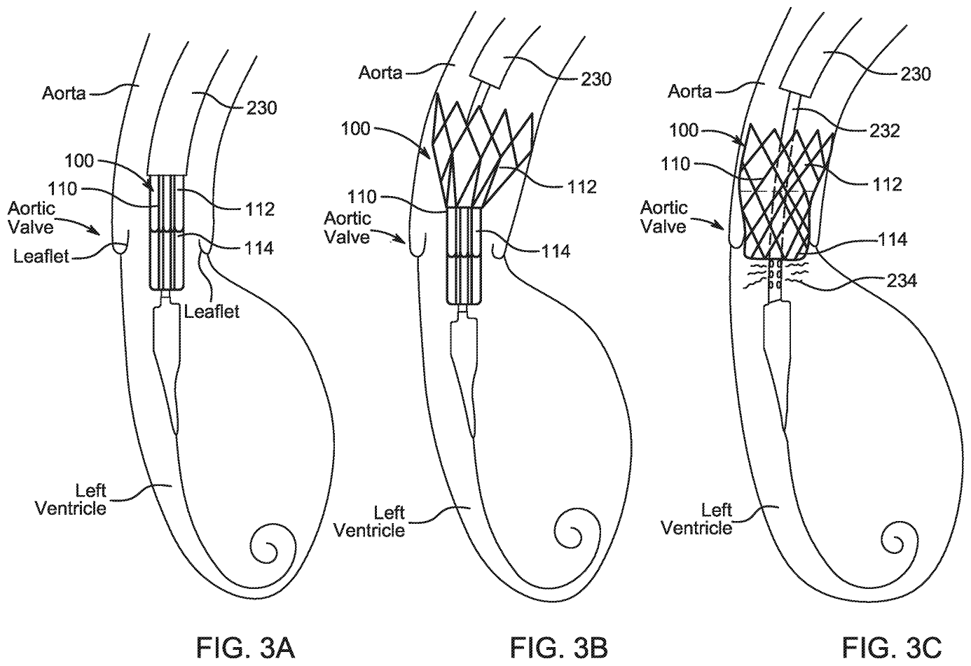

FIGS. 3A-3C illustrate a method of implanting the expandable device 100 using a retrograde approach. It will be appreciated that the expandable devices 100 of the present technology may be delivered to the native valve region using other approaches, such as antegrade, trans-septal, or trans-apical approaches. As shown in FIGS. 3A-3C, the expandable device 100 can be intravascularly delivered to a desired location in the heart, such as an intracardiac location near the aortic valve, while in the delivery (e.g., collapsed) configuration within a delivery catheter or sheath 230. The device 100 can be advanced to a position in which the heat-expandable portion 114 is within or upstream of the plane of the native annulus, as shown in FIG. 3A. The sheath 230 may then be withdrawn proximally beyond the heat-expandable portion 114 and the self-expanding portion 112, thereby releasing the self-expanding portion 112 such that it self-expands into apposition with tissue at or downstream of the native annulus, as shown in FIGS. 3B and 3C. As depicted in FIG. 3C, a fluid 234 having a second temperature greater than the first temperature may be infused at or upstream of the heat-expandable portion 114 via an elongated shaft 232 extending through the lumen of the heat-expandable portion 114. Upon reaching the second temperature, the heat-expandable portion 114 expands into its austenite shape in apposition with adjacent tissue. In contrast to balloon expandable devices, the expandable device 100 of the present technology expands without the use of a balloon and thus avoids the associated challenges of aortic occlusion during deployment. However, the heat-expandable portion could be expanded via a balloon or other mechanical expansion mechanism if so desired. Once positioned, the expandable device 100 may then be detached from the delivery system, and the delivery system may be removed from the patient.

In any of the foregoing embodiments, should the heat-expandable portion need to be repositioned or re-shaped (for example, in response to the dynamic environment of the heart and/or dilation of the annulus over time), the heat-expandable portion 114 may be re-heated to the second temperature (at or above A_{f}) to re-shape and/or reposition the device.

Although the foregoing description of the expandable device 100 and anchoring member 110 is made with reference to aortic valve replacement, it will be appreciated that the expandable device 100 and/or anchoring member 110 may be used for aortic valve repair, mitral valve repair, and/or mitral valve replacement.

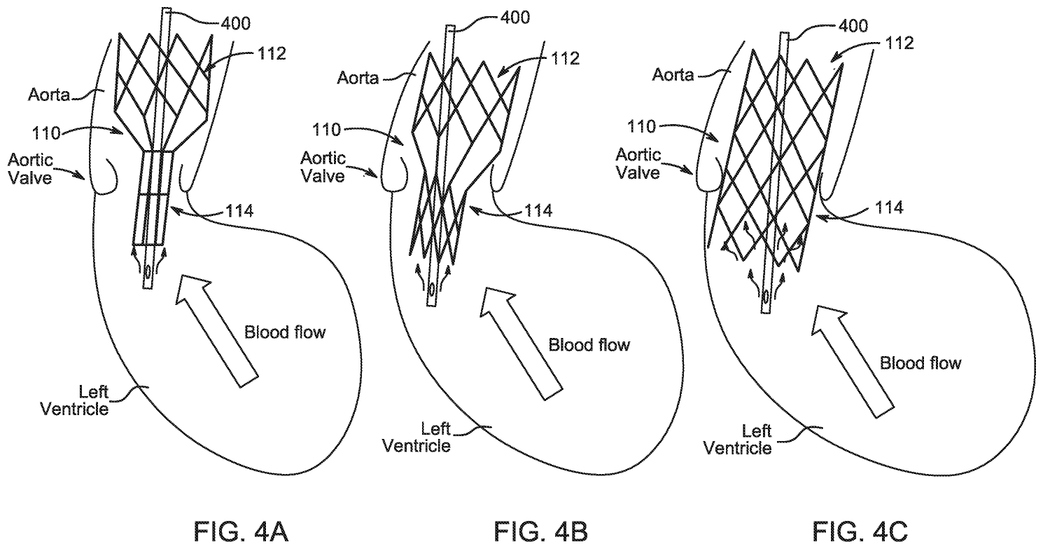

FIGS. 4A-7C show various devices, systems, and methods for delivering thermal energy to the anchoring member 110 (or heat-expandable portion 114 thereof) to transform the heat-expandable portion 114 from its martensite state to its austenite or “remembered” shape. FIGS. 4A-4C illustrate a method for expanding the second portion 114 via infusion of a warm fluid distal of the anchoring member 110 and/or heat-expandable portion 114 such that the ventricular blood flow carries the warm fluid proximally through and along the heat-expandable portion 114 and/or the rest of the device 100. The fluid may be infused at a second temperature greater than the first temperature.

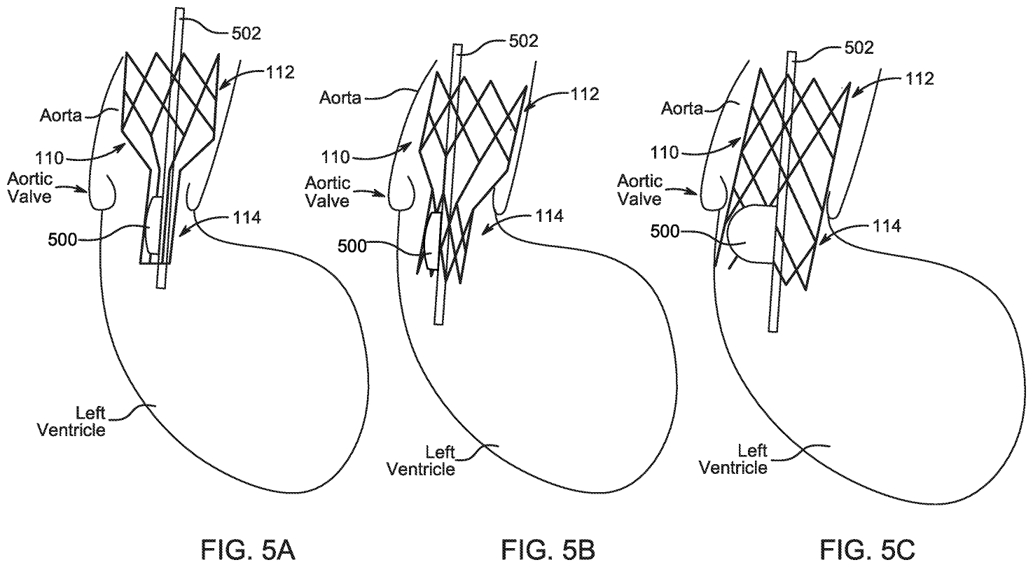

FIGS. 5A-5C illustrate a method for expanding the heat-expandable portion 114 via infusion of a fluid (liquid or gas) to an inflatable member 500 positioned within the lumen of the heat-expandable portion 114. In some embodiments, for example, the inflatable member 500 may be a balloon. In some embodiments, the inflatable member 500 may expand around less than 360 degrees of the circumference of the heat-expandable portion 114 (such as an eccentric balloon), thereby allowing blood flow through the aortic annulus during the deployment procedure. In those embodiments using an omni-directional balloon, the balloon may be inflated and deflated quickly (in a matter of a few seconds) to avoid aortic occlusion. The balloon may be simply inflated once with a warm liquid such as saline to achieve the desired warming effect, or the balloon might have two or more lumens filling it from the catheter, so that warm saline can be circulated continuously to gradually heat the balloon to a specific desired temperature which is warm enough to expand the stent, but not warm enough to damage the valve or the surrounding tissue. The balloon might alternatively have an electric circuit to heat the balloon. The balloon might also have a thermistor to measure the temperature of the balloon.

If the balloon is used to heat just one portion of the stent, then the balloon catheter may be steerable to press it against one wall or the other, or it may have an expanding bow or strut on the side of the catheter opposite the balloon to help press it against the desired portion of the stent.

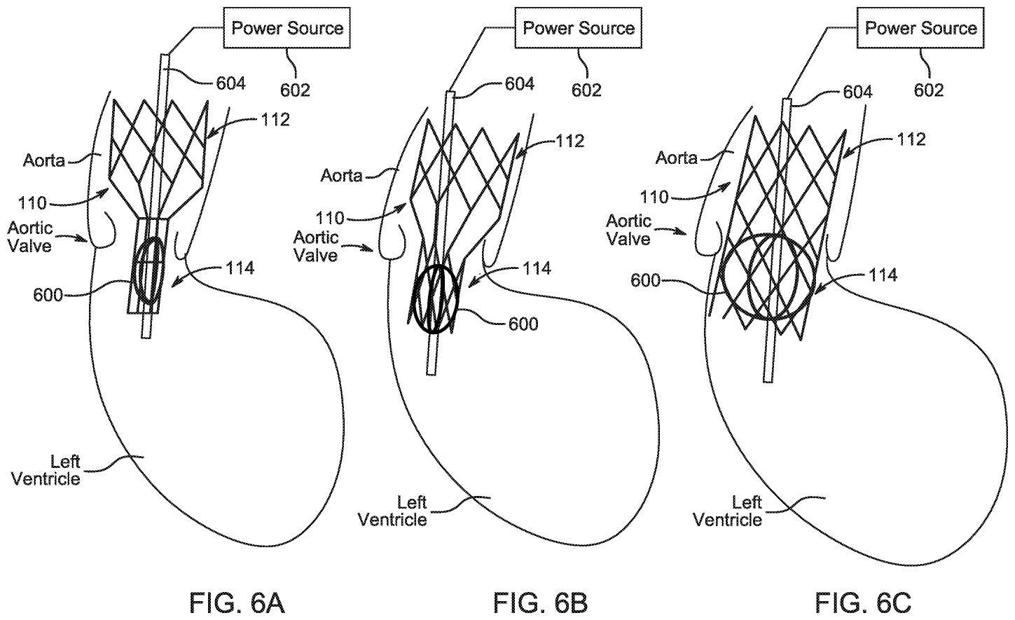

FIGS. 6A-6C illustrate a method for expanding the heat-expandable portion 114 via an expandable heating element 600. The heating element 600 may be made from a plurality of struts. At least a portion of each of the struts may include an electrically conductive material. The heating element 600 may be coupled to a power source 602 located at a proximal, extracorporeally-positioned portion of the treatment system via an elongated conduction element 604. When activated, the power source 602 heats the struts of the heating element 600, which radiate heat in the direction of the heat-expandable portion 114. In some embodiments, the entirety of the heating element 600 may be heated and/or electrically conductive. In other embodiments, only portions of the heating element 600 and/or struts of the heating element 600 may be electrically conductive and/or portions of the heating element 600 and/or struts may be insulated.

The heating element 600 may be configured to self-expand, or may expand in response to the thermal energy applied by the power source 602. In an expanded state, the struts may assume a basket-like shape that is generally spherical, cylindrical, spheroid, ovoid, or other suitable shapes. The heating element 600 may expand into contact with the heat-expandable portion 114, thereby directly transmitting thermal energy to the heat-expandable portion 114 to expand the heat-expandable portion 114. As the heating element 600 expands, it may push radially outwardly against an inner surface of the heat-expandable portion 114, thereby forcing the heat-expandable portion 114 to expand. The heating element 600 may also have thermal insulation on the inside of the struts so that blood flow doesn't cool the stent as quickly as it is heated, and so that the heating elements don't need to be heated to a very high temperature to generate a meaningful increase in stent temperature.

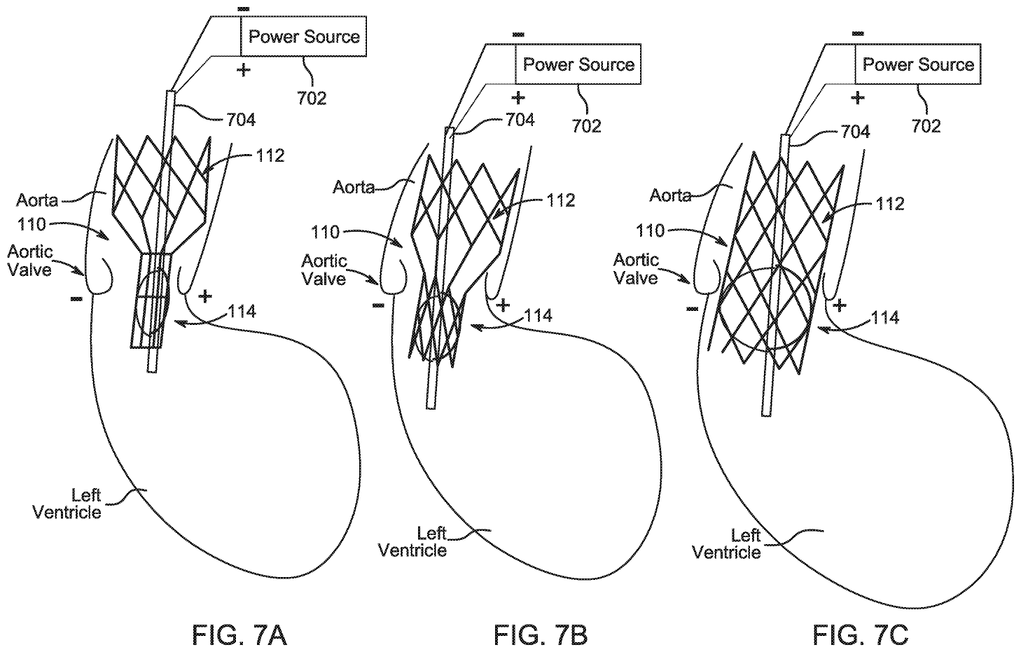

FIGS. 7A-7C illustrate a method for expanding the heat-expandable portion 114 via heat delivered directly to the metallic structure of the heat-expandable portion 114. For example, all or a portion of the heat-expandable portion 114 may be formed with an electrically conductive material, and the anchoring member 110 and/or heat-expandable portion 114 may be coupled to an extracorporeally-positioned power source 702 via an elongated conduction member 704.

The structure of the heat-expandable portion 114 of the expandable devices 100 disclosed herein are considerably more structurally robust than typical self-expanding stent structures. A typical self-expanding stent wants to return to its fully expanded state when confined within a catheter at a first temperature (for example, average body temperature). This persistent chronic outward force against the inner surface of the catheter lumen can make it very difficult to move the catheter wall relative to the stent. As such, one of the design constraints of typical self-expanding stents is chronic outward force. The expandable devices 100 of the present technology, however, are not limited by this constraint and can have struts that have stiff cross sectional geometries in the bending planes, wider strut angles, etc. Increasing the stiffness of the heat-expandable portion 114 may be especially beneficial because the martensitic SMA will have a Young's Modulus that is considerably lower than stainless steel or cobalt chromium (the materials that balloon-expandable valves are typically made from).

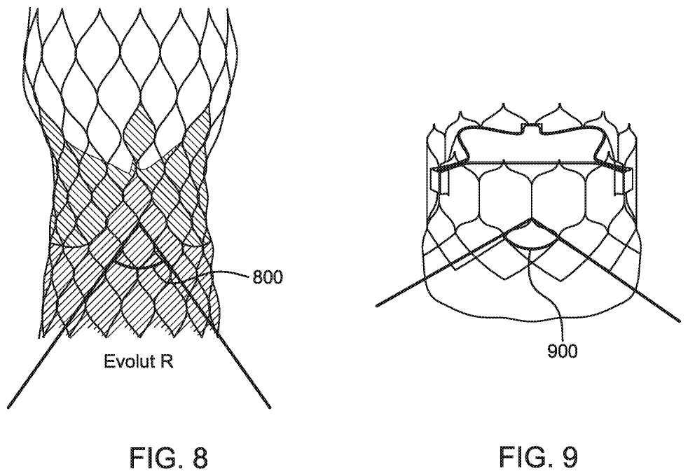

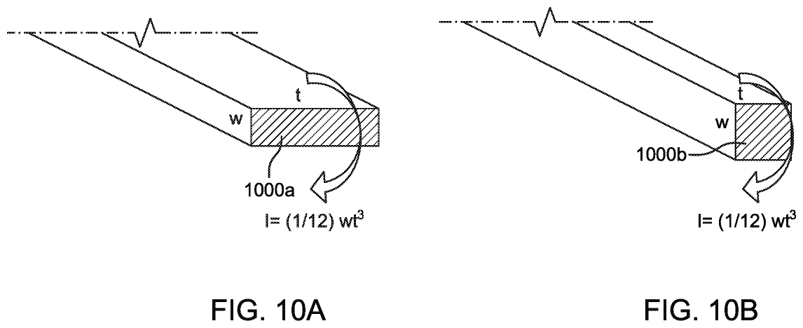

The strut angles can be greater in balloon-expandable stent structures as compared to self-expanding structures, as shown by angles 900 and 800 in FIGS. 9 and 8, respectively. This is because the balloon-expandable structures are plastically deformed whereas the self-expanding structures need to be elastically deformed during compression and then bounce back to their original shapes. For example, FIGS. 10A and 10B show cross-sections of struts used in typical self-expanding and balloon-expandable structures, respectively. The moment of inertia is heavily dependent on width w, but the strain on the material is also greatly affected. In order to keep the material within its elastic strain limit but add overall stiffness, the struts of typical self-expandable stents have a width w that is significantly less than its thickness t. The respective thicknesses t of the struts can be measured along a line orthogonal to and extending radially from a central longitudinal axis of the expandable device and/or anchoring member when the expandable device and/or anchoring member is considered in a tubular shape (e.g., the perpendicular distance between the respective strut's luminal and abluminal surface), or as a dimension that is orthogonal to a plane of the expandable device and/or anchoring member when represented as laid-flat. The respective widths of the struts can be measured as the distance that is generally orthogonal to the thickness t.

As evident in FIG. 10B, the struts of balloon-expandable structures trend more towards a square cross-section as the expansion mechanism depends on plastic deformation. Balloon-expandable stents generally have more design freedom to optimize cross-sections to minimize material used to get the stiffness desired. The martensitic properties of the expandable devices 100 of the present technology allow for a structural design similar to balloon-expandable devices because the expandable device 100 does not need to be compressed into a low-profile, delivery configuration in its superelastic state (when T>A_{f}). Rather, the expandable device 100 may be compressed in its easily deformable martensite state and expanded to its austenite state with the application of heat.

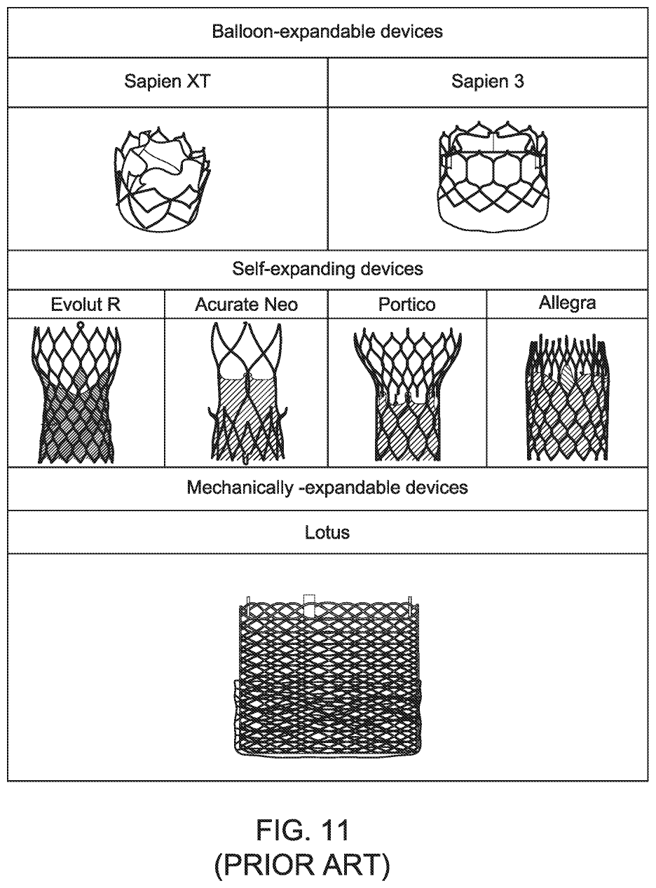

Additional examples of conventional balloon-expandable and self-expanding stents are shown in FIG. 11, as well as a mechanically-expandable stent.

As previously mentioned, the expandable device 100 may include a valve 120 coupled to or configured to be coupled to the anchoring member 110. The valve 120 may comprise a temporary or permanent valve adapted to block blood flow in the upstream direction and allow blood flow in the downstream direction. In some embodiments, the valve 120 may be a replacement valve configured to be disposed in the expandable device 100 (or component thereof) after the device 100 is implanted at the native valve. The valve 120 can have a plurality of leaflets, and may be formed of various flexible and impermeable materials including PTFE, Dacron®, pyrolytic carbon, or other biocompatible materials or biologic tissue such as pericardial tissue or xenograft valve tissue such as porcine heart tissue or bovine pericardium.

In some embodiments, the expandable device 100 optionally includes a valve support (not shown) positioned at least partially within the anchoring member 110. In such embodiments, the prosthetic valve 120 is coupled to, mounted within, or otherwise carried by the valve support. The device 100 may further include one or more sealing members (not shown) and/or tissue engaging elements (not shown). The sealing member can extend around an inner wall of the anchoring member 110 to prevent paravalvular (e.g., paraprosthetic) leaks between the device 100 and the native tissue and/or between the anchoring member 110 and the valve support (if included). In some embodiments, the tissue engaging elements can be spikes disposed on an outer surface of the anchoring member 110 (along one or both of the self-expanding and heat-expandable portions 112 and 114) and extend in an angled and/or radially outward direction to engage, and in some embodiments, penetrate the native tissue to facilitate retention or maintain position of the device 100 in a desired implanted location.

### CONCLUSION

Although many of the embodiments are described above with respect to devices, systems, and methods for replacing and/or repairing an aortic valve, the technology is applicable to other applications and/or other approaches, such as repair and/or replacement of a mitral valve or any other native valve in the mammalian body. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1-11.

The above detailed descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1. An anchor member configured to be positioned at a treatment site proximate a native valve annulus of a human patient, the anchor member comprising:
an expandable structure comprising a first portion and a second portion, each having a low-profile state and an expanded state, wherein, when the expandable structure is positioned at the treatment site at a first temperature of no greater than about 40° C. and released from a catheter:
the first portion self-expands toward its expanded state and into apposition with tissue at or near the annulus to secure the anchor member at the treatment site, and
the second portion remains in its low-profile state,
wherein the second portion of the expandable structure is configured to expand into apposition with tissue at or near the annulus when heated to a second temperature greater than the first temperature.

2. The anchor member of claim 1, wherein the second portion is formed of a shape memory alloy (“SMA”) having an austenite finish temperature A_{f} that is: (a) greater than or equal to the second temperature, and (a) greater than body temperature.

3. The anchor member of claim 1, wherein the second temperature is no less than 40° C.

4. The anchor member of claim 1, wherein the second temperature is from about 37° C. to about 40° C.

5. The anchor member of claim 1, wherein the second portion is formed of an SMA having a martensite finish temperature M_{f} greater than or equal to the first temperature and an austenite finish temperature A_{f} less than or equal to the second temperature.

6. The anchor member of claim 1, wherein the second portion is formed of an SMA having a martensite start temperature Mₛ greater than or equal to the first temperature and an austenite finish temperature A_{f} less than or equal to the second temperature.

7. The anchor member of claim 1, wherein the second portion is formed of an SMA having:
a martensite finish temperature M_{f} greater than or equal to the first temperature,
a martensite start temperature Mₛ greater than or equal to the first temperature, and
an austenite finish temperature A_{f} less than or equal to the second temperature.

8. The anchor member of claim 1, wherein the second portion is formed of an SMA having:
a martensite finish temperature M_{f} less than the first temperature,
a martensite start temperature Mₛ greater than or equal to the first temperature, and
an austenite finish temperature A_{f} less than or equal to the second temperature.

9. The anchor member claim 1, wherein the second portion has an austenite finish temperature A_{f} less than 37° C.

10. The anchor member of claim 1, wherein the expandable structure is configured such that, when implanted at the native valve annulus, the second portion is upstream of the first portion.

11. The anchor member of claim 1, wherein the expandable structure is configured such that, when implanted at or near a native aortic valve annulus, (a) at least a portion of the first portion is positioned within the aorta and, (b) at least a portion of the second portion is positioned within the left ventricle.

12. The anchor member of claim 1, wherein the expandable structure is configured such that, when implanted at or near a native aortic valve annulus, no portion of the first portion is pressing outwardly against the left ventricle.

13. The anchor member of claim 1, wherein the expandable structure is configured such that, when implanted at or near an annulus of an aortic valve of the patient, no portion of the first portion is distal of the annulus.

14. The anchor member of claim 1, wherein, when the expandable structure is implanted at or near a native aortic valve annulus such that both the first and second portions are expanded and in contact with adjacent tissue, the first portion presses outwardly against adjacent tissue with greater force than the second portion presses outwardly against adjacent tissue.

15. The anchor member of claim 1, wherein the second portion is heat-expandable.

16. The anchor member of claim 1, wherein the second portion does not self-expand at or below the second temperature.

17. An expandable device configured to be positioned at an implantation site proximate a native valve annulus, the expandable device comprising:
an anchoring member comprising:
an expandable structure comprising a first portion and a second portion,
wherein, when the first portion is positioned at the implantation site at a first temperature and released from a constrained delivery state, the first portion is configured to self-expand into apposition with tissue at or near the annulus to secure the anchor member at the implantation site, and
wherein the second portion remains in a low-profile state at the first temperature and is configured to expand into apposition with tissue at or near the annulus when heated to a second temperature greater than the first temperature secure the anchor member at the implantation site; and
a prosthetic valve configured to be carried by, mounted within, or coupled to the anchoring member.

18. A system for treating a native cardiac valve of a human patient, the system comprising:
a sheath;
an anchoring member configured to be delivered through the sheath to a treatment site proximate a native valve annulus, the anchoring member comprising:
an expandable structure comprising a first portion and a second portion,
wherein, when the first portion is positioned at the treatment site at a first temperature and released from a constrained delivery state, the first portion is configured to self-expand into apposition with tissue at or near the annulus to secure the anchor member at the treatment site, and
wherein the second portion remains in a low-profile state at the first temperature and is configured to expand into apposition with tissue at or near the annulus when heated to a second temperature greater than the first temperature;
a prosthetic valve configured to be carried by, mounted within, or coupled to the anchoring member;
an elongated member having a proximal end portion configured to be positioned at an extracorporeal location during implantation of the expandable structure and a distal end portion configured to be delivered through the sheath to the treatment site; and
a heating element carried by the distal end portion of the elongated member, wherein the heating element is configured to facilitate heating of the second portion to the second temperature.

19. The system of claim 18, wherein the heating element is a plurality of openings in the distal end portion of the elongated member, and wherein the system further comprises a fluid source coupled to the proximal end portion of the elongated member and configured to deliver heated fluid through a lumen extending through the elongated member and through the openings to a vicinity of the second portion.

20. The system of claim 18, wherein the heating element is a balloon carried by the distal end portion of the elongated member, and wherein the system further comprises a fluid source coupled to the proximal end portion of the elongated member and configured to deliver heated fluid to the balloon through a lumen extending through the elongated member.

21. The system of claim 18, wherein the heating element is an expandable basket carried by the distal end portion of the elongated member, and wherein the system further comprises a power source coupled to the elongated member, and wherein at least a portion of the elongated member is electrically conductive such that, when the power source is activated, the elongated member transfers energy to the basket, thereby heating and expanding the basket.

22. The system of claim 18, wherein the heating element is an electrode that is in direct contact with the second portion, and wherein applying current to the elongated member passes current through the second portion, thereby heating the second portion and causing it to expand.
